(19) United States
(12) Patent Application Publication
(10) Pub. No.: US 2025/0235127 A1
(43) Pub. Date: Jul. 24, 2025

(54) SIGNAL QUALITY ASSESSEMENT SYSTEM AND BLOOD GLUCOSE LEVEL PREDICTION SYSTEM BASED ON PHOTOPLETHYSMOGRAPHY

(71) Applicant: Intelligent Information Security Technology Inc., Hsinchu (TW)

(72) Inventors: Wai-Chi Fang, Hsinchu (TW); Chia-Yu Liao, Hsinchu (TW); Ching-Jung Lin, Hsinchu (TW)

(21) Appl. No.: 18/669,473

(22) Filed: May 20, 2024

Related U.S. Application Data
(60) Provisional application No. 63/623,797, filed on Jan. 22, 2024.

(51) Int. Cl. A61B 5/145; A61B 5/00; A61B 5/1455

(52) U.S. Cl. CPC A61B 5/14532; A61B 5/1455; A61B 5/7246; A61B 5/725; A61B 5/7267; A61B 5/742

(57) ABSTRACT

The present invention relates to a signal quality assessment system and a blood glucose level prediction system based on photoplethysmography. The signal quality assessment system comprises a near-infrared light photoplethysmography sensor, a Bluetooth module, an AI acceleration platform, and an electronic device. The AI acceleration platform serves as the central analysis unit, receiving PPG signals from the front-end sensing unit via an interface. Signal processing, including bandpass filtering composed of high-pass and low-pass filters and data normalization, is executed by the processor. Configurable AI chips are used to evaluate the quality of PPG signals, facilitating the discard of unwanted signals and subsequent reconstruction for blood glucose assessment. Processed PPG signals and the corresponding assessed blood glucose values are transmitted to a display. The display provides a comprehensive and real-time view of the monitored data, enabling efficient and timely interpretation of blood glucose values.

### CROSS-REFERENCE TO RELATED APPLICATIONS

The present application claims the benefit of U.S. Provisional Patent Application No. 63/623,797 filed on Jan. 22, 2024. The contents of all of the aforementioned applications are incorporated by reference herein in their entirety.

### TECHNICAL FIELD

The present invention relates to a signal quality assessment system and a blood glucose level prediction system based on photoplethysmography, in particular to a signal quality assessment system and a blood glucose level prediction system for evaluating blood glucose concentration by analyzing variations in physiological signals.

### BACKGROUND

The rapid development of society has also led to various lifestyle-related diseases, with diabetes being recognized as a prominent chronic illness. According to the International Diabetes Federation's statistical report in 2021, a staggering 5.37 billion adults worldwide, constituting approximately 8% of the global population, were living with diabetes. Three quarters of these individuals with diabetes reside in low to middle income countries. Projections estimate that the diabetic population will grow to 643 million by 2030 and continue to increase, reaching an alarming 783 million by 2045. Factors such as the prevalence of fast-food culture, refined sugar consumption, and sedentary lifestyles have become invisible catalysts for the growing diabetes epidemic.

In the pursuit of improving blood glucose monitoring for individuals with diabetes, noninvasive blood glucose research has emerged as a prominent and widely explored area of study. Various methods fall under the umbrella of noninvasive blood glucose research, broadly categorized as skin impedance analysis, tear component analysis, reverse iontophoresis, and optical spectroscopy techniques. Impedance Spectroscopy, utilizing multiple electrodes, tracks changes in tissue impedance influenced by blood sugar levels. However, the contact impedance between the skin and electrodes poses challenges to measurement accuracy. Tear fluid spectral analysis, employing specialized contact lenses, aims to measure glucose concentrations in tears. Yet, it is crucial to acknowledge limitations, such as potential discomfort from tear stimulation and the fact that glucose levels in tears may not precisely mirror blood glucose concentrations. The reverse iontophoresis method involves passing microcurrents across the skin surface, causing movement of ions and neutral glucose molecules to estimate blood sugar levels. However, this method is not without drawbacks, including skin irritation, the necessity for a minimum duration for obtaining blood glucose measurements, and impracticality during periods of patient sweating. Optical techniques, such as Raman spectroscopy, delve into the chemical reactions of glucose molecules with specific light wavelengths. While this method has been used to estimate glucose concentration, challenges like laser instability, varying light intensity, extended acquisition times, and impractical measuring devices hinder its development. Another optical technique employed for the development of noninvasive blood glucose level monitoring is Near-infrared (NIR) spectroscopy.

In conclusion, in order to overcome the aforementioned shortcomings, the inventors of the present application have devoted significant research and development efforts and spirit, continuously breaking through and innovating in the present field. It is hoped that novel technological means can be used to address the deficiencies in practice, not only bringing about better products to society but also promoting industrial development.

### SUMMARY

To achieve the above-mentioned objectives, the present invention provides a signal quality assessment system based on photoplethysmography (PPG), comprising: a database, an AI acceleration platform, and an electronic device. The database includes a blood glucose level dataset and a PPG signal dataset. The blood glucose dataset includes at least one blood glucose value, and the PPG signal dataset includes at least one PPG signal. The PPG signal is obtained through a PPG sensor detecting light absorption and reflection rates from the blood of a subject.

In an embodiment of the present invention, the AI acceleration platform is communicatively connected to the database through a Bluetooth module, and the AI accelerator platform comprising: a first processor, a preprocessing engine, and an AI module. Firstly, the first processor is communicatively connected to the database, and the first processor obtains a training dataset from the blood glucose dataset and a PPG signal dataset. Secondly, the preprocessing engine is communicatively connected to the first processor through a first interface and extracts multiple PPG feature maps from the PPG signal in the training dataset.

In an embodiment of the present invention, the AI module comprises multiple layers of convolutional neural network and is communicatively connected to the first processor through the first interface. The AI module is trained according to the PPG feature maps and using a template matching method and a peak detection method to extract multiple pulse waveforms from the PPG feature maps, and a template PPG signal is established. A correlation coefficient is calculated between the template PPG signal and the pulse waveform. Each time the AI module is trained to generate the correlation coefficient, a quality classification label, and an initial training model, and the correlation coefficients are averaged to obtain a threshold value. Further, when the correlation coefficient is greater than or equal to the threshold value, the training of the AI module is completed to generate a PPG signal quality assessment module, the quality classification label and the PPG signal quality assessment module are transmitted to the first processor through the first interface.

In an embodiment of the present invention, the electronic device is communicatively connected to the first processor through a transmitter, and the electronic device is configured to display the quality classification labels including vectors [0,1] and [1,0], and the PPG signal quality assessment module.

In an embodiment of the present invention, the preprocessing engine includes a bandpass filter and a data normalization calculator, the bandpass filter filters the PPG signal in the range of 0.5 Hz to 5 Hz to eliminate noise interference outside the frequency range of 0.5 Hz to 5 Hz and an external high-frequency noise to produce bandpass-filtered data. Further, the data normalization calculator normalizes the bandpass-filtered data input every 5 seconds using following equation 1, where x[n]ϵX and n=1, 2 to 320,

$$y [ n ] = \frac{x [ n ] - min ( x )}{max ( x ) - min ( x )} . \qquad equation 1$$

In an embodiment of the present invention, the timelines from both the blood glucose level dataset and the PPG feature maps were extracted and compared to align at the same time points through the template matching method. Further, the blood glucose values were retained after alignment, and the corresponding PPG signals were selected for one-minute records.

In an embodiment of the present invention, the extracted one-minute PPG feature maps are subdivided into 5-second windows, the pulse waveform within a window is extracted by using the peak detection, the template PPG signal is constructed by averaging each pulse waveform within a window, and the correlation coefficient between the template PPG signal and the pulse waveform is calculated according to the following Formula 2,

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} {\sum }_{i = 1}^{n} {( {y}_{i} - \overline{y} )}^{2}}} . \qquad equation 2$$

In an embodiment of the present invention, a difference value between the quality classification label and a true label is generated, the difference value is calculated by a loss function, specifically a binary cross entropy, and the weights of each layer of convolutional neural networks are iteratively adjusted based on the loss function.

To achieve the anther objective, the present invention provides a blood glucose level prediction system, comprising: a dataset, a second processor, an edge AI accelerator, and an electronic device. The dataset includes at least one PPG signal with a quality classification label represented by vector [0,1] generated by the aforementioned signal quality assessment system based on photoplethysmography. The second processor is communicatively connected to the dataset through a second interface and obtains a blood glucose training dataset from the dataset.

In an embodiment of the present invention, the edge AI accelerator is communicatively connected to the second processor through the second interface to receive the PPG signals from the blood glucose training dataset to accomplish a blood glucose prediction result. The edge AI accelerator comprising: a data memory module, a processing element array module, a convolutional neural network accelerator module, and a long short term memory module. Firstly, the data memory module stores the PPG signal and computes multiple PPG feature maps, and relevant instructions are provided to the second processor for resetting or inputting data. Secondly, the processing element array module is composed of multiple processing units, each processing unit includes a multiply accumulate unit to handle convolution and matrix-vector multiplication operations, and the PPG signal performs parallel processing. Further, the convolutional neural network accelerator module includes multiple convolutional layers, a pooling layer, a fully connected layer, and multiple first memories used to store weights required by the convolutional layers, the pooling layer, and fully connected layers. The PPG signal is accelerated and processed in parallel through each processing unit to generate the PPG feature maps, and the PPG feature maps are processed through the activation function to a generate convolutional pooling data. The convolutional pooling data is then stored in the first memories for subsequent layer calculations, or sent back to the second processor for convolutional pooling data verification and analysis. In addition, the convolutional pooling data is recursively processed through the long short term memory module, and the convolutional pooling data is computed in each processing unit to extract temporal features to generate recursive data. Next, a regression prediction is performed by the fully connected layer, and the recursive data undergoes matrix-vector product computation in each processing unit to generate a predicted blood glucose value and a blood glucose prediction module. The predicted blood glucose value and the blood glucose prediction module are transmitted to the second processor through the second interface.

In an embodiment of the present invention, the electronic device is communicatively connected to the second processor through a transmitter, and the electronic device is configured to display the predicted blood glucose value and the blood glucose prediction module.

In an embodiment of the present invention, the long short term memory module includes multiple second memories storing the weights corresponding to the PPG feature maps and the weights of hidden states, the long short term memory network module performs a gated parameter operation on the convolutional pooling data, and the operation is carried out in each processing unit to generate recursive data which is then processed through the activation functions to calculate the final current state (Ct) value and the hidden state (Ht) value.

In an embodiment of the present invention, each convolutional layer includes at least one filter and uses a Rectified Linear Unit function as a first activation function. Further, the fully connected layer includes at least one neuron and uses a linear function as a second activation function, and a regression prediction is performed through the fully connected layer.

In an embodiment of the present invention, the linear function includes both the Rectified Linear Unit function and a Softmax function. When a preceding layer of the fully connected layer is not the final layer, the fully connected layer uses the Rectified Linear Unit function as the activation function, and when the fully connected layer is the final layer, the second activation function becomes Softmax, which the result of the Rectified Linear Unit function can be directly determined by utilizing signed bits to output to the first memories.

In an embodiment of the present invention, a difference value between the predicted blood glucose value and a true blood glucose value is generated, the difference value is calculated by a loss function, specifically the Mean Square Error (MSE), and the weights of each layer of convolutional neural network accelerator module are iteratively adjusted based on the loss function.

### BRIEF DESCRIPTION OF THE DRAWINGS

FIG. 1 is a schematic diagram of a signal quality assessment system of the present invention.

FIG. 2 is a system architecture diagram of the signal quality assessment system of the present invention.

FIG. 3 is a flowchart of a template matching method of the present invention.

FIG. 4 is a flowchart of a So and Chan method of the present invention.

FIG. 5 is a bar chart of the parameters and accuracy of different numbers of filters in the convolutional neural network layer of the present invention.

FIG. 6 is a bar chart of the impact of normalization on model performance in LOSOV validation of the present invention.

FIG. 7 is an accuracy curve chart of impact of normalization on model performance of the present invention.

FIG. 8 is a loss curve chart of impact of normalization on model performance of the present invention.

FIG. 9a is a curve chart of unacceptable PPG segment before normalization of the present invention.

FIG. 9b is a curve chart of unacceptable PPG segment after normalization of the present invention.

FIG. 10 is a system architecture diagram of an edge AI accelerator of the present invention.

FIG. 11 is a schematic diagram of two types of signal inputs for the processing element array module of the present invention.

FIG. 12 is a schematic diagram of the processing element array module of the present invention.

FIG. 13 is a schematic diagram of the convolutional neural network accelerator module of the present invention.

FIG. 14 is a schematic diagram of the long short term memory network module of the present invention.

FIG. 15 is a schematic diagram of the matrix-vector product unit of the present invention.

FIG. 16 is a curve chart of mean absolute error within 250 cycles of the present invention.

FIG. 17 is a curve chart of the mean square error within 250 cycles of the present invention.

FIG. 18 is an error bars analysis between predicted blood glucose level concentrations and reference blood glucose concentrations on the test data of the present invention.

FIG. 19 is a Clarke error grid analysis of the blood glucose evaluation model of the present invention.

### DETAILED DESCRIPTION

In order to enable a person skilled in the art to better understand the objectives, technical features, and advantages of the invention and to implement it, the invention is further elucidated by accompanying appended drawings. This specifically clarifies the technical features and embodiments of the invention, and enumerates exemplary scenarios. To convey the meaning related to the features of the invention, the corresponding drawings herein below are not and do not need to be completely drawn according to the actual situation. Throughout the text, PPG refers to Photoplethysmography (PPG).

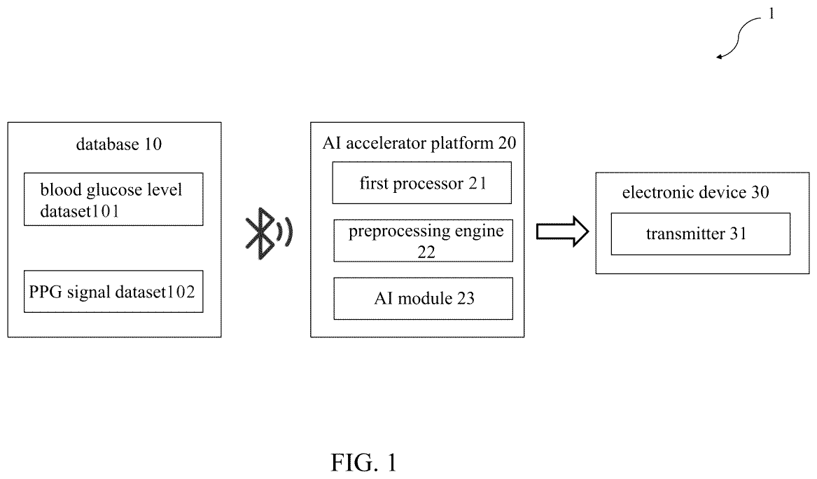

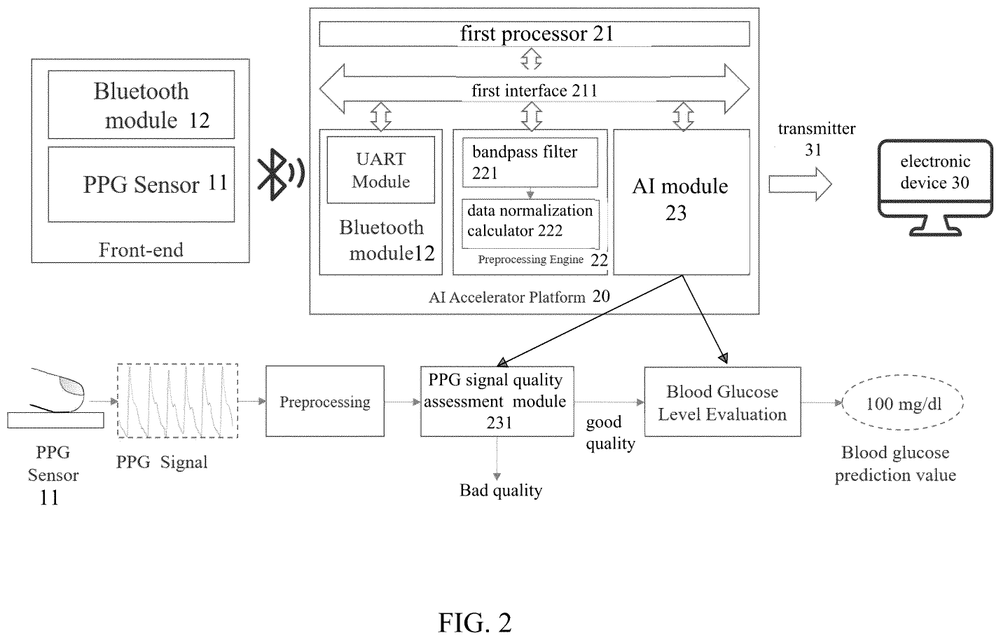

Please refer to FIG. 1 and FIG. 2, FIG. 1 is a schematic diagram of a signal quality assessment system of the present invention; and FIG. 2 is a system architecture diagram of the signal quality assessment system of the present invention.

As shown in FIGS. 1 and 2, the present invention provides a signal quality assessment system 1 based on photoplethysmography (PPG), comprising: a database 10, an AI acceleration platform 20, and an electronic device 30. The database 10 includes a blood glucose level dataset 101 and a PPG signal dataset 102. The blood glucose dataset 101 includes at least one blood glucose value, and the PPG signal dataset 102 includes at least one PPG signal. The PPG signal is obtained through a PPG sensor 11 detecting light absorption and reflection rates from the blood of a subject.

The PPG sensor 11 is a photoplethysmography (PPG) sensor that utilizes near-infrared light, precisely designed for noninvasive blood glucose monitoring. Photoplethysmography (PPG) captures variations in light as it traverses the skin, tissues, and blood vessels. The morphology of the PPG is primarily influenced by heart activity, with the systolic period causing the first peak and the diastolic period resulting in the second wave peak. In addition to heart activity, other factors affecting PPG light absorption or reflection, such as molecular components in the blood, the quantity of oxygen carrying red blood cells, and respiration, contribute to changes in PPG morphology. Consequently, the PPG signal offers insights into vital signs, including heart rate, blood pressure, blood oxygen concentration, fatigue detection, and diabetes identification.

The blood glucose values and PPG signals used in the present invention are obtained from public websites. After downloading the blood glucose values and PPG signals, records with corresponding blood glucose values and PPG signals at the same time points are extracted as valid data, while invalid records containing only blood glucose values or only PPG signal records are excluded. A public dataset obtained from Physionet, and a cohort of 16 post-menopausal female patients, aged between 35 and 65 years, was recruited for a prospective study conducted at the Duke Endocrinology and Lipids Clinic. Participants were selected based on having either high normal blood glucose (HbAlc 5.2˜5.6) or prediabetes (HbAlc 5.7˜6.4). Exclusion criteria encompassed conditions such as cancer, COPD, cardiovascular disease, food allergies, and any use of antidiabetic drugs. For data collection, participants were instructed to wear a continuous glucose monitor (CGM; Dexcom G6) and a noninvasive wearable smartwatch (Empatica E4) continuously for a duration of 8˜10 days. The Dexcom G6 recorded interstitial glucose concentration (mg/dL) every 5 minutes, while the Empatica E4 featured sensors for photoplethysmography (optical heart rate) sampled at 64 Hz, electrodermal activity (galvanic skin response, related to sweat activity) sampled at 4 Hz, skin temperature sampled at 4 Hz, and triaxial accelerometry sampled at 32 Hz.

The AI accelerator platform 20 is communicatively connected to the database 10 through a Bluetooth module 12, and the AI module 20 operates as the central analytical unit in the signal quality assessment system 1. The Bluetooth module 12 is commercially available IC modules, which are incorporated for enhanced efficiency and reliability, facilitating the integration and sensing of the PPG signal. Furthermore, the AI accelerator platform 20 comprises a first processor 21, a preprocessing engine 22, and an AI module 23; wherein the first processor 21 is communicatively connected to the database 10, and the first processor 21 is a RISC-V (Reduced Instruction Set Computing-V) processor that orchestrates the data flow. The first processor 21 retrieves the valid data from the blood glucose dataset 101 and the PPG signal dataset 102. The valid dataset is then divided into 10 parts, with one part used as the test dataset and the remaining 9 parts split into 80% for training dataset and 20% for validation dataset. The process is repeated 10 times to validate the accuracy performance of the AI module 23.

The preprocessing engine 22 is communicatively connected to the first processor 21 through a first interface 211, and the first interface 211 is an advanced Peripheral Bus. Further, the preprocessing engine 22 includes a bandpass filter 221 and a data normalization calculator 222, and extracts multiple PPG feature maps from the PPG signal in the training dataset. The bandpass filter 221 is comprised of a high-pass filter with a cutoff frequency of 0.5 Hz and a low-pass filter with a cutoff frequency of 5 Hz. The bandpass filter 221 filters the PPG signal in the range of 0.5 Hz to 5 Hz to eliminate noise interference outside the frequency range and an external high-frequency noise to produce bandpass-filtered data; wherein the range of 0.5 Hz to 5 Hz is widely recognized as the appropriate frequency band range for the PPG signal in the human body. The noise interference includes low-frequency noise such as baseline drift or respiratory interference and high-frequency noise stemming from external electrical field interference or suboptimal contact. After the bandpass filter 221, the PPG signal is subjected by the data normalization calculator 222, ensuring that comparisons are not skewed by inherent differences in baseline values among subjects. The data normalization calculator 222 provides a more reliable basis, as the baseline of the PPG signal varies among each subject. Normalizing the PPG signal helps to eliminate differences among participants. Specifically, the data normalization calculator normalizing the bandpass-filtered data input every 5 seconds using following equation 1, where x[n]ϵX and n=1, 2 to 320.

$$y [ n ] = \frac{x [ n ] - min ( x )}{max ( x ) - min ( x )} . \qquad equation 1$$

While the undesirable PPG signals can be eliminated through high-pass and low-pass filtering, PPG signals with severe deformations and significant amplitude variations are still retained. Therefore, it is necessary to exclude or reconstruct these severely deformed PPG signals. To overcome the above-mentioned issues, the signal quality assessment system 1 of the present invention utilizes the AI module 23 to screen the quality of the PPG signals, thereby retaining high quality PPG signals for training a blood glucose prediction module.

The AI module 23 comprises multiple layers of convolutional neural network including multiple convolutional layers, a pooling layer, a fully connected layer, and is communicatively connected to the first processor 21 through the first interface 211. The AI module 23 is trained according to the PPG feature maps and using a template matching method and a peak detection method to extract multiple pulse waveforms from the PPG feature maps, and a template PPG signal is established. A correlation coefficient is calculated between the template PPG signal and the pulse waveform. Each time the AI module 23 is trained to generate the correlation coefficient, a quality classification label, and an initial training model, and the correlation coefficients are averaged to obtain a threshold value. Further, when the correlation coefficient is greater than or equal to the threshold value, the training of the AI module 23 is completed to generate a PPG signal quality assessment module 231, the quality classification label and the PPG signal quality assessment module are transmitted to the first processor 21 through the first interface 211.

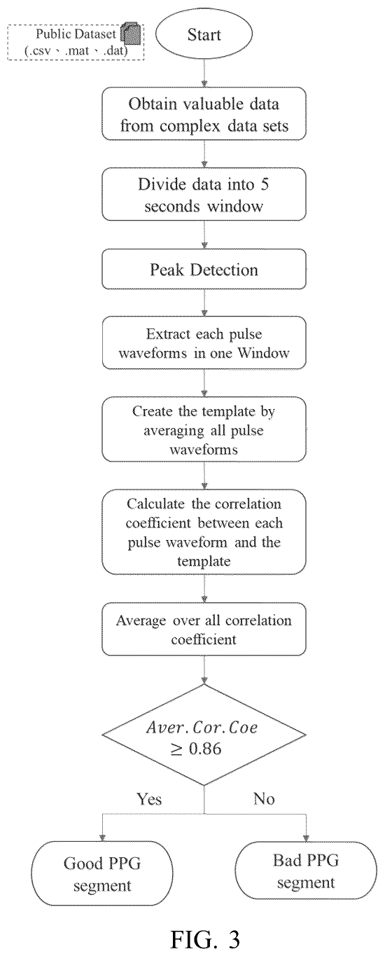

Please refer to FIG. 3, FIG. 3 is a flowchart of a template matching method of the present invention.

As shown in FIG. 3, the “pandas” Python package is employed to read and import the dataset in .csv format. The timelines from both the blood glucose level dataset and the PPG feature maps were extracted and compared to align at the same time points through the template matching method, and blood glucose values were retained after alignment, and the corresponding PPG signals were selected for one-minute records. The template matching method facilitated the acquisition of labeled PPG signals with associated BGL (blood glucose level) values, serving as input for a blood glucose level prediction system. Namely, extracting valuable data from complex datasets. Then, the extracted one-minute PPG signal data is subdivided into 5-second windows for quality assessment. Next, each pulse waveform is extracted within a window through the peak detection method. The correlation coefficient between the template PPG signal and the pulse waveform is calculated according to the following Formula 2.

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} {\sum }_{i = 1}^{n} {( {y}_{i} - \overline{y} )}^{2}}} . \qquad equation 2$$

Following the method proposed by C. Orphanidou, T. Bonnici, P. Charlton, D. Clifton, D. Vallance, and L. Tarassenko (IEEE Journal of Biomedical and Health Informatics, Vol. 19, May 2015, pp. 832-838), an average threshold value of 0.86 is suggested. The correlation coefficient is used, the quality of PPG labels is classified as “good” or “poor”. The threshold value greater than or equal to the threshold are considered “good” quality, while those below the threshold value are classified as “poor” quality.

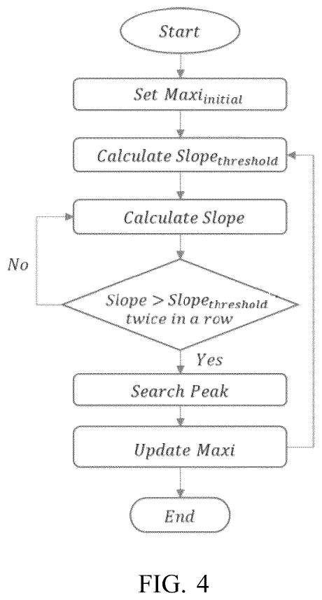

Please refer to FIG. 4, FIG. 4 is a flowchart of a So and Chan method of the present invention. The template matching method employs the “So-and-Chan” algorithm to detect the peak point, as shown in FIG. 4. In the initial step, the initial maximum value is determined by selecting the maximum amplitude among the first one-second samples of the PPG signal. Subsequently, the threshold value for the slope is obtained by dividing the initial maximum value by 2. The next step involves slope calculation using the equation 3, which corresponds to the convolution of the neighborhood of 2 points with the vector [−2, 1, 1, 2].

$$Slpoe [ n ] = ( - 2 ) \cdot x [ n - 2 ] - x [ n - 1 ] + x [ n + 1 ] + 2 \cdot x [ n + 2 ] . \qquad equation 3$$

The slope threshold value and slope value are determined, the comparison between these two values commences. When the slope value surpasses the slope threshold value twice consecutively, the pulse onset is identified. The peak point is then located by finding the next instance where the slope becomes smaller than or equal to zero. Finally, the indices and amplitudes of both the onset and peak are acquired. These four values are utilized to update the maximum value through equation 4, subsequently refining the slope threshold for the next iteration of peak detection.

$${Maxi}_{new} = \frac{{Peak}_{height} - {Onset}_{height} - Maxi}{2} + Maxi . \qquad equation 4$$

The electronic device 30 is communicatively connected to the first processor 21 through a transmitter 31. The electronic device 30 is configured to display the quality classification labels including vectors [0,1] and [1,0], and the PPG signal quality assessment module; wherein [0,1] is set as good quality, and [1,0] as poor quality. The processed PPG signals and corresponding assessed blood glucose values are transmitted to the electronic device 30 in real-time through the UART protocol. The electronic device 30 provides a comprehensive and instantaneous view of the monitored data, enabling efficient and timely interpretation of blood glucose values.

To determine the most effective parameters for the AI module 23 while keeping the filter size fixed at 3 by 1. The initial configuration involves initializing the number of filters in each convolutional layer, ranging from 3 to 10. Additionally, the max pooling layer is combined with a 1-dimensional convolutional layer featuring a pool size of 2 by 1 and a stride step of 2, using the Rectified Linear Unit (ReLU) activation function. The PPG quality classification layer utilizes the softmax activation function. The Adam optimizer, as introduced by Kingma and Ba is employed, seamlessly integrating both momentum and adaptive learning rate mechanisms. The learning rate is set to 0.0001, and binary entropy serves as the loss function, given by equation 5. To prevent the AI module 23 overfitting, early stopping is implemented, halting training when the validation loss does not improve within 15 epochs. A difference value between the quality classification label and a true label is generated, the difference value is calculated by a loss function, specifically a binary cross entropy, and the weights of each layer of convolutional neural networks are iteratively adjusted based on the loss function.

$$loss function = - \frac{1}{N} {\sum }_{i = 1}^{N} log ( {p}_{i} ) \qquad equation 5$$

The momentum mechanism accelerates gradient updates when the previous gradient shares the same “sign” as the current gradient, enhancing model convergence. Conversely, it slows down gradient changes when the previous gradient has the opposite “sign”, allowing for a more careful descent towards minimum gradients as they decrease. The momentum mechanism proves instrumental in achieving smoother convergence. The adaptive learning rate mechanism shares a conceptual foundation with momentum. It dynamically adjusts the learning rate based on the average of previous gradients. When the average of all previous gradients is small, the learning rate is increased to expedite gradient descent.

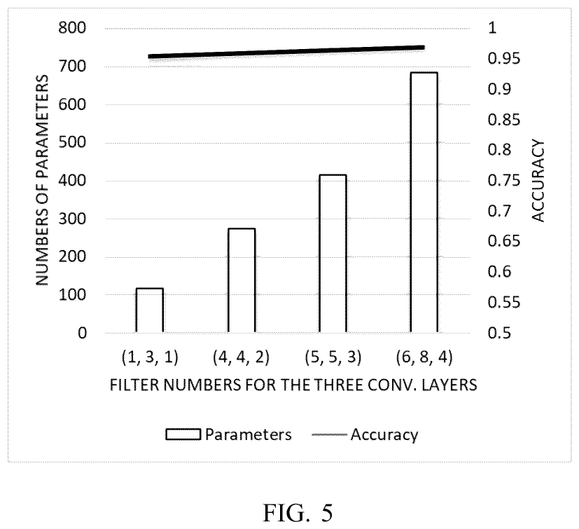

Please refer to FIG. 5, FIG. 5 is a bar chart of the parameters and accuracy of different numbers of filters in the convolutional neural network layer of the present invention. Through systematic iteration, the optimal parameters for the model are identified, which entail 6 filters in the first layer, 8 filters in the second layer, and 4 filters in the third layer, achieving the highest accuracy performance of 97.18%. In the initial optimization with filter numbers of (6,8,4) for the three convolutional layers, the model requires 684 parameters (2736 bytes of space, where 1 parameter requires 4 bytes of space) for storage, achieving an accuracy of 97.18%. In one embodiment, after the final optimization by removing filters with zero outcomes, an optimized configuration with 1 filter in the first layer, 3 filters in the second layer, and 1 filter in the third layer is achieved. The configuration requires 116 parameters (464 bytes of space) for storage, with an accuracy of 95.59%. With a minor sacrifice in accuracy (1%), the parameters are significantly reduced by a factor of 6. Compared to a three-layer Convolutional Neural Network, networks with fewer layers still result in a higher number of parameters. The presence of convolutional layers and max pooling layers contributes to data dimension reduction, a factor directly associated with a decrease in the number of parameters needed.

The raw PPG data undergoes bandpass filter 221 and passes through the PPG signal quality assessment module 231 to retain clear PPG signals suitable for training the BGL assessment model. The PPG signal quality assessment module 231 identifies distinct PPG segments, which are then reshaped into one-second-length samples to effectively obtain BGL predictions in real-world scenarios. Due to the extraction of lower-dimensional feature maps with additional convolutional layers, the AI module 23 is confined to four convolutional layers. This restriction stems from the initial input, a 1-dimensional image of size 64 with a convolutional filter size of (3,1). Expanding beyond four layers becomes impractical as the feature map's shape from the fourth max-pooling layer reduces to 2, smaller than the convolutional filter shape of 3. The AI module 23 comprises four convolutional layers with filter numbers set to 64, 128, 256, and 256, respectively, and Rectified Linear Unit (ReLU) as the activation function. Additionally, the AI module 23 includes two dense layers with 1024 and 1 neurons, respectively. These dense layers are specifically designed for regression prediction, providing users with an interpretable Blood Glucose Level (BGL) value.

In one embodiment, the decrease in layer numbers is determined by the existence of zero features across the PPG data corresponding to various BGL situations. Following the approach, the model's hyperparameters are systematically tuned, while also considering changes in errors. The Adam optimizer is employed with a learning rate set to 0.0001, utilizing mean square error (MSE) as the loss function and serving as an indicator of the AI module 23 accuracy. To effectively reduce the parameters in accordance with the pruning strategy, the number of neurons is reduced in the fully connected layer. The number of neurons in this layer significantly impacts the overall parameter count, as each neuron connects to every input feature. The required parameters can be computed as inputₛₕₐₚₑ×Neurons_{number}+Neurons_{number}, including biases. However, the reduction results in an increase in errors, rendering the AI module's performance unacceptably low. Given the priority of the BGL prediction model to achieve high accuracy and reliability, the focus is on tuning the convolutional layers. As a result, the number of parameters decreases through the reduction in one layer and later increases as the influence of other layers on AI module 23 performance is assessed.

To determine the final optimal hyperparameters that strike a balance between the number of parameters and errors in the signal quality assessment system 1. The detailed configuration of the AI module 23 is outlined in Table 1, featuring 32 filters for the first Convolutional (Conv.) layer, 64 filters for the second Conv. layer, 128 filters for the third Conv. layer, 128 filters for the fourth Conv. layer, 256 neurons for the first fully connected (FC) layer, and 1 neuron for the output FC layer. The activation functions employed in this architecture are Rectified Linear Unit (ReLU) for Conv. layers and linear for FC layers. The ultimate optimized model demonstrates notable performance, achieving a Mean Absolute Error (MAE) of 5.541 and MSE of 101.115 with a model complexity represented by 147,073 parameters. In contrast, the AI module 23 yields an MAE of 4.875 and MSE of 88.573, yet requires a significantly larger parameter count, totaling 848,129. The comparative analysis underscores a substantial reduction in the number of parameters, approximately a sixfold decrease, as a result of the proposed model tuning from the baseline.

TABLE 1

| Layer | Filter size | Filter Number | Stride | Activation Function |
|---|---|---|---|---|
| Conv. layer | (3, 1) | 32 | 1 | Relu |
| Max pooling | (2, 1) | | 2 | |
| Conv. layer | (3, 1) | 64 | 1 | Relu |
| Max pooling | (2, 1) | | 2 | |
| Conv. layer | (3, 1) | 128 | 1 | Relu |
| Max pooling | (2, 1) | | 2 | |
| Conv. layer | (3, 1) | 128 | 1 | Relu |
| Max pooling | (2, 1) | | 2 | |
| Max pooling | (2, 1) | | 2 | |
| Dense | | 256 | | linear |
| Dense | | 1 | | linear |

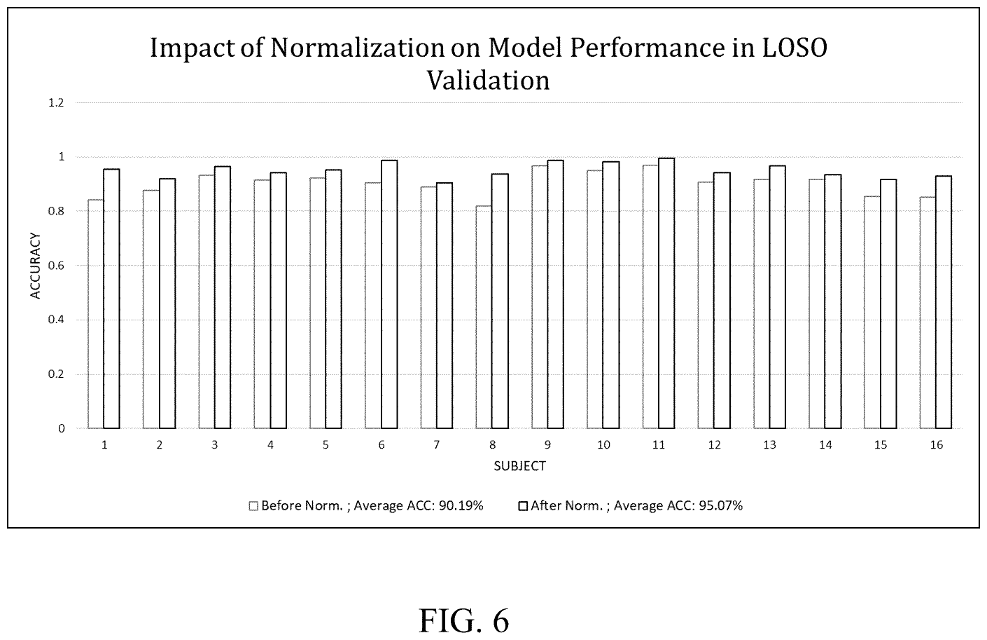

Please refer to FIG. 6, FIG. 6 is a bar chart of the impact of normalization on model performance in LOSOV validation of the present invention. To analyze the impact of data normalization, the leave-one-subject-out validation (LOSOV) method is employed to investigate its influence across subjects. The LOSOV results, depicted in FIG. 6, indicate an overall improvement in accuracy across all individual subjects with the implementation of data normalization; wherein each subject consists of two bar charts, with the left side showing the data before normalization, and the right side showing the data after normalization. Notably, there are striking enhancements for specific subjects, such as a 11% improvement for the PPG quality assessment model of subject 1 and a 12% improvement for subject 8. These substantial improvements, raising accuracy from approximately 80% to over than 90% for these two subjects, underscore the essential nature of data normalization.

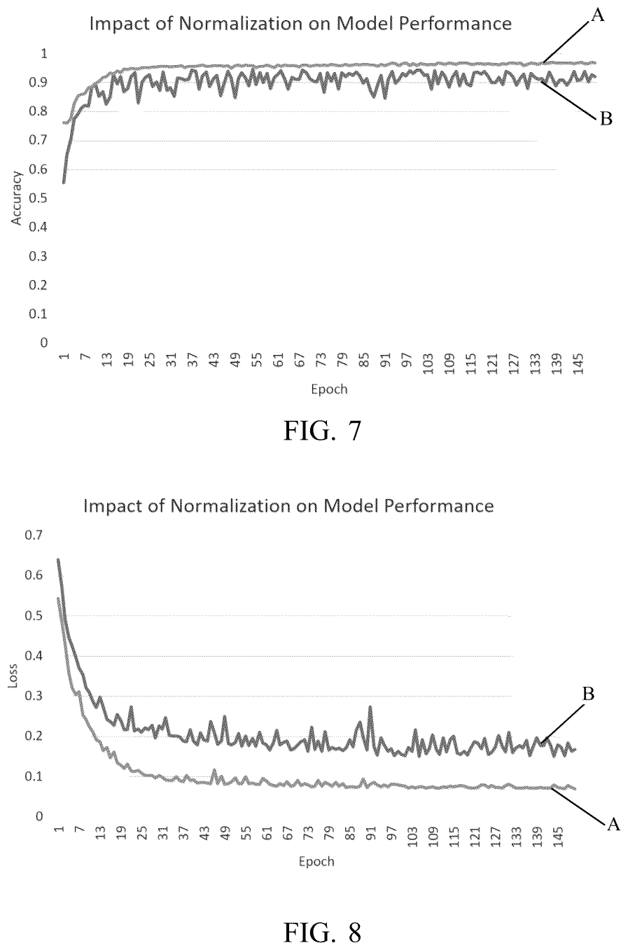

Please refer to FIG. 7 and FIG. 8, FIG. 7 is an accuracy curve chart of impact of normalization on model performance of the present invention; and FIG. 8 is a loss curve chart of impact of normalization on model performance of the present invention. In FIGS. 7 and 8, two curves are generated over 150 epochs. Compared to the curves with normalization (A line), noticeable oscillations in the validation accuracy and loss curves (B line) are observed. These oscillations stem from certain subjects with higher or lower baseline values disproportionately influencing the results. Such bias could limit the model's applicability to a broader population.

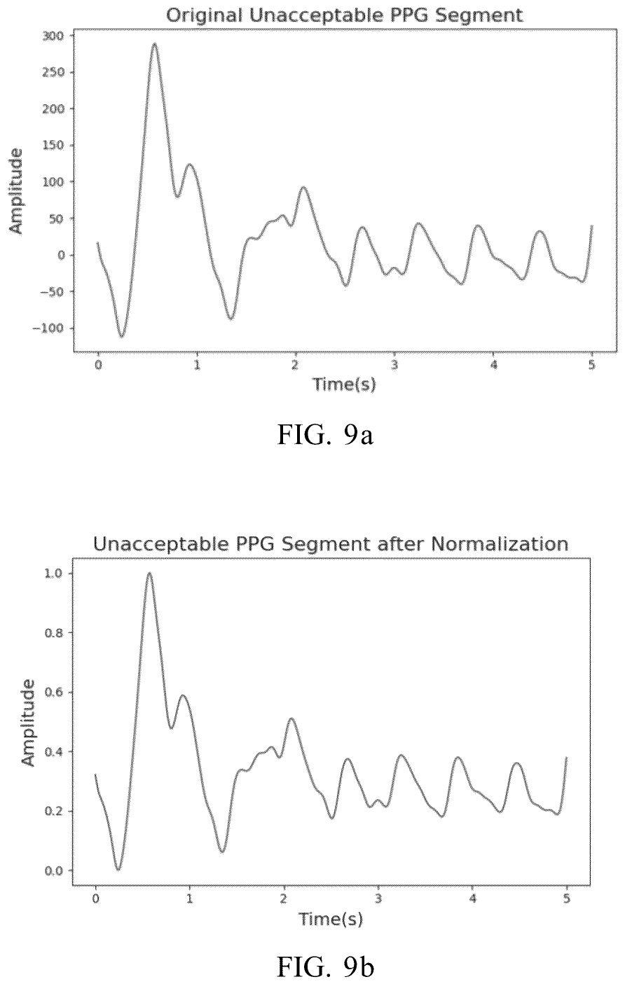

Please refer to FIG. 9a and FIG. 9b, FIG. 9a is a curve chart of unacceptable PPG segment before normalization of the present invention; and FIG. 9b is a curve chart of unacceptable PPG segment after normalization of the present invention. Another perspective to validate the accuracy improvement by data normalization is to directly analyze cases where the AI module 23 trained on the original dataset incorrectly predicts “Good” quality when the real quality is “Bad,” while the model trained on the normalized dataset correctly predicts “Bad” quality. In FIGS. 9a and 9b, unacceptable segments before and after data normalization are displayed. Although they have a highly similar appearance, the model predictions are completely opposite. In a 2-class classification with “Good” quality represented as (1,0) and “Bad” quality as (0,1), the output of Softmax was (0.8331, 0.1669) without data normalization, leading to the incorrect identification of these samples as “Good” quality. With data normalization, the output was (0.0053, 0.9947), correctly predicting “Bad” quality.

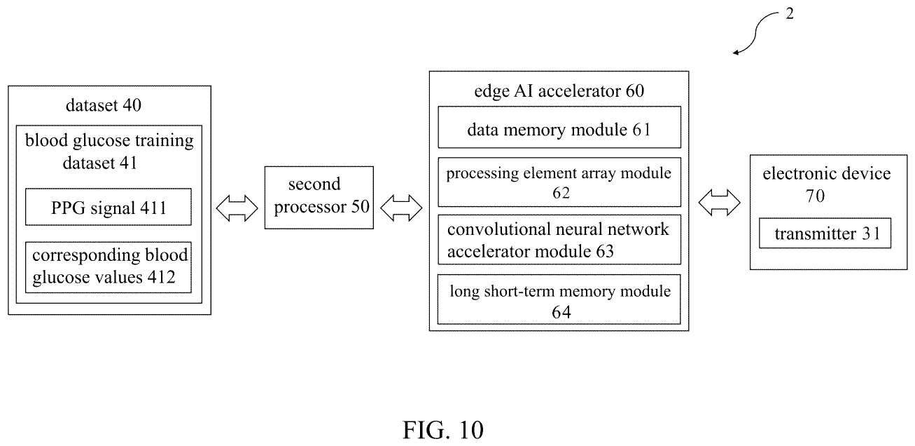

Please refer to FIG. 10, FIG. 10 is a system architecture diagram of an edge AI accelerator of the present invention. a blood glucose level prediction system, comprising: a dataset 40, a second processor 50, an edge AI accelerator 60, and an electronic device 70.

The dataset 40 including at least one PPG signal with a quality classification label represented by vector [0,1], generated by the aforementioned signal quality assessment system based on photoplethysmography. The PPG signal 411 and its corresponding blood glucose values 412 for the quality classification label are split into 10 parts. One part was selected as the testing dataset, while the remaining nine parts were divided into 80% for training data and 20% for validation data. This process was repeated 10 times to validate the model's accuracy performance The input to the blood glucose level prediction system 2 is the PPG signal for good quality, where the quality classification label for the PPG signal is represented as the vector [0,1]. Further, the second processor 50 is an RISC-V (Reduced Instruction Set Computing-V) processor that orchestrates the data flow. The second processor 50 is responsible for controlling the blood glucose level prediction system 2, ensuring seamless coordination among the components. The second processor 50 is connected to the dataset 40 through a second interface 511, which communicatively connected to the Advanced Peripheral Bus (APB). The second processor 50 retrieves a blood glucose training dataset 41 from the dataset 40, which includes the PPG signal 411 and the corresponding blood glucose values 412.

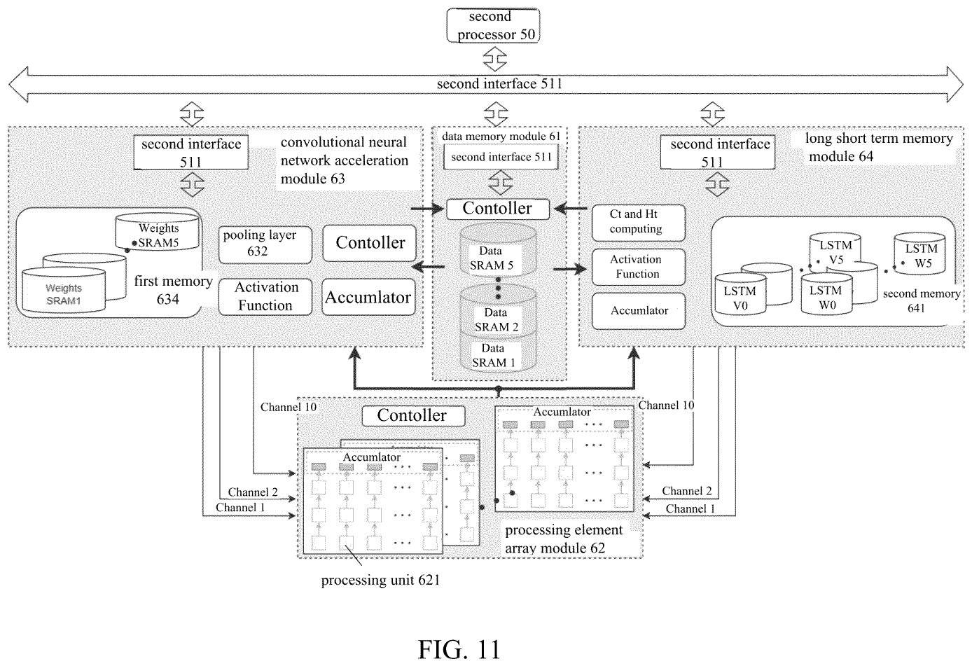

Please refer to FIG. 11, FIG. 11 is a schematic diagram of two types of signal inputs for the processing element array module of the present invention.

As shown in FIG. 11, the edge AI accelerator 60 communicatively connected to the second processor 50 through the second interface 511 to receive the PPG signals from the blood glucose training dataset to accomplish a blood glucose prediction result, and the edge AI accelerator 60 comprising: a data memory module 61, a processing element array module 62, a convolutional neural network accelerator module 63, and a long short term memory module 64.

The data memory module 61 stores the PPG signal 411 and the computed multiple PPG feature maps 413, and provides relevant instructions to the second processor 50 for resetting or inputting data. The data memory module 61 is composed of 5 1024×32 double-word Static Random Access Memory (SRAM), responsible for feature map storage, including the initial input images and the extracted features between each layer. The related instructions are built to instruct data input or memory reset.

Next, the processing element array module 62 is composed of multiple processing units 621, each containing a multiplier accumulator unit 6211 to handle convolution and matrix-vector multiplication operations, and to parallel process the PPG signal 411. In a preferred embodiment of the present invention, the second processor 50 utilizes 10 processing units 621 to form the processing element array module 62. The utilization of the processing element array module 62 brings substantial enhancement, especially in the convolutional neural network acceleration module 63 and the processing element array module 62. The convolutional neural network acceleration module 63 and the processing element array module 62 demand extensive multiply and accumulation calculations, and the integration of the processing element array module 62 contributes significantly to the overall computational efficiency and performance. The data length is defined as 8 bits for integers and 8 bits for floating-point numbers, with each considered as one word in the edge AI accelerator 60. The edge AI accelerator 60 to handle not only convolution computing but also matrix-vector product computations. The design innovation empowers the processing element array module 62 to efficiently manage the majority of computational workloads within AI computing. To further optimize computing efficiency, the processing element array module 62 is expanded to include 10 units, facilitating the parallel processing of 10-channel input data. Each processing units 621 effectively controls the data flow and partitions large input data into the appropriate format to match the available computing tolerance at any given time.

Control signals and essential hyperparameters needed to configure the customized edge AI accelerator 60 are transmitted from the second processor 50 to the designated computing module through the Advanced Peripheral Bus (APB) protocol. The communication protocol facilitates efficient data exchange, enabling the second processor 50 to dynamically adjust and optimize the edge AI accelerator 60 based on specific computational requirements. The operation instructions include assigning the appropriate slave module (ID) to receive subsequent instructions, inputting images and weights to store in data memory and computing modules, respectively, setting hyperparameters for customized configuration, specifying the SRAM read start address to instruct the computing module to access the correct data in SRAM, and outputting results for obtaining the computing results.

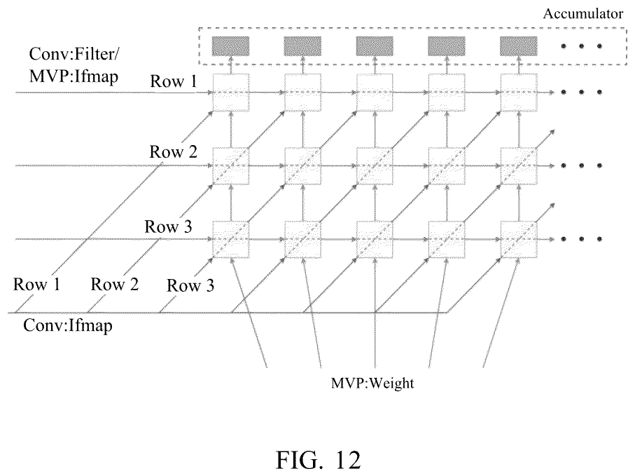

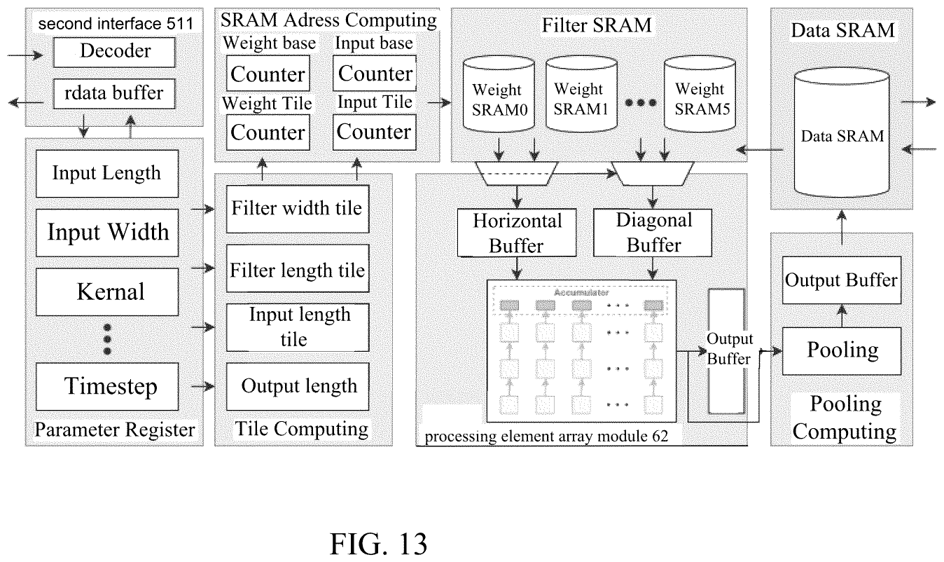

Please refer to FIG. 12, FIG. 12 is a schematic diagram of the processing element array module of the present invention. As shown in FIGS. 11 and 12, the processing element array module 62 consists of multiple processing units 621. Each processing element array module 62 contains 3×10 processing units 621, capable of processing a 3×3 filter and a 3×12 input feature map (Ifmap) simultaneously. The processing element array module 62 proposes two signal input methods: horizontal broadcasting (horizontal data flows A1, A2, A3 in FIG. 12) and diagonal broadcasting (diagonal data flows B1, B2, B3 in FIG. 12). The broadcasting methods enable input data to be simultaneously received by the processing units 621 for multiplication and addition operations within one cycle. The results are then shifted vertically using shift registers to the upper layer of the processing units 621 for the next cycle of multiplication and addition, ultimately passing through accumulators to obtain the convolution operation result. Additionally, the processing element array module 62 adjusted the matrix-vector product (MVP) operation by introducing vertical shift input, wherein the input is passed upward through shift registers once per cycle, rather than broadcasting. The processing element array module 62 requires only minimal additional hardware, such as MUX, to enable hardware reuse for both LSTM and dense layer operations using the same processing element array module 62. Therefore, in the implemented hardware architecture, the two most computationally demanding operations in CNN and LSTM into the same hardware structure are integrated. During convolution, the filter is input through horizontal broadcasting, while input feature map is input through diagonal broadcasting. During matrix-vector product computation, weights are input sequentially through vertical shift registers, and input feature map is input through horizontal broadcasting.

The arrangement of the processing element array module 62 consists of grouping every three processing units 621 to form a column, where the calculated values accumulate upwards. The third row of the processing units 621 is simplified, skipping unnecessary adders to save a cycle and reduce processing time. Each horizontal and diagonal input undergoes broadcasting through the register buffer, while vertical input comes from the bottommost PE. The PE array structure includes 10 row inputs, employing a shift register configuration. The top layer of the PE array is an accumulator, capable of simultaneously accumulating values for the entire column and the external pseudo-sum.

The convolutional neural network accelerator module 63 includes multiple convolutional layers 631, a pooling layer 632, a fully connected layer 633, and multiple first memories 634 used to store weights required by the convolutional layers 631, the pooling layer 632, and fully connected layers 633. The PPG signal 411 is accelerated and processed in parallel through each processing unit to generate the PPG feature maps 413. Further, the PPG feature maps 413 is processed through the activation function to a generate convolutional pooling data, which is then stored in the first memories 634 for subsequent layer calculations, or sent back to the second processor 50 for convolutional pooling data verification and analysis.

The convolutional layers 631 is utilizes diagonal broadcasting input and horizontal broadcasting input, excluding the input from the vertical shift register as it is reserved for matrix-vector product computation. In the convolution operation, both the filter and input feature maps are broadcasted horizontally and diagonally to the processing element array module 62. Due to row stationary, each cycle can complete the product of one row of the filter and one row of the input feature map. To prevent partial sums between the two sets of inputs from influencing each other, a cycle with zero input is inserted between two convolution operations, setting the input to zero. Therefore, each convolutional layer 631 operation requires W+1 cycles, where W is the width of the filter.

Additionally, Tile-based and Zero-padding methods are employed to handle diverse inputs that may be too large or too small, including input feature maps and filter. This enables the execution of inputs with different shapes on the fixed hardware structure, eliminating the need for extra hardware costs to accommodate various scenarios. In the computation, the hardware processing order involves handling the Tile and zero-padding of the filter first, followed by the processing of the Tile and zero-padding of input feature maps. Any filter not meeting the height of three pixels is zero-padded. While zero-padding is also applied to width, during actual computation, rows with zeros are skipped to reduce processing time. Parts exceeding three pixels in dimensions are cut for batch processing. Since the processing engine array has control hardware for zero input, the zero-padding method optimizes the control unit of convolution with minimal additional power consumption.

The width of input feature maps requires no processing due to the row stationary algorithm. Heights exceeding 12 pixels are cut, and tile-based overlap is applied based on the filter height. Portions with a height less than 12 use zero-padding to fill with zeros. When stored back to data SRAM, the zero-padding part is skipped to avoid wasting time and SRAM space.

Prior to computation, the aforementioned Tile-based and zero-padding algorithms are executed through the Tile computing module. Subsequently, the SRAM address computing module maps the results of these two algorithms to the correct addresses for storing the filter and input feature map in SRAM. The data is then read from SRAM, transmitted to the PE array for convolution, processed by the pooling module, and finally, stored back in data SRAM for further processing by the next layer. In the present invention, the pooling layer computation employs max pooling, leveraging its widespread adoption in various models. A pooling kernel size of 2×2 or 2×1 is specifically chosen. The selection of the size is automatic and depends on whether the user's input feature map is a two-dimensional or one-dimensional vector. Consequently, the computation process is streamlined, achieved through the implementation of straightforward shifters and comparators.

Please refer to FIG. 13, FIG. 13 is a schematic diagram of the convolutional neural network accelerator module of the present invention. As shown in FIG. 13, the instructions received from the second interface 511 undergo decoding and profiling to extract the necessary information such as weights, start read address, and hyperparameters essential for the long short term memory module 64 computations. The vector inner product is executed on the processing element array module 62, utilizing existing hardware components like adders and multiplexers to minimize the chip size and enhance power efficiency. The long short term memory module 64 recursively processes the convolutional pooling data in each processing unit 621. It computes the convolutional pooling data to extract temporal features, generating recursive data for regression prediction to be performed by the fully connected layer 633. The recursive data undergoes matrix-vector product computation in each processing unit 621 to generate a predicted blood glucose value and a blood glucose prediction module. The predicted blood glucose value and the blood glucose prediction module are then transmitted to the second processor 50 through the second interface 511.

Further, the electronic device 70 is communicatively connected to the second processor 50 through a transmitter, and configured to display the predicted blood glucose value and the blood glucose prediction module. The long short term memory module 64 consists of multiple second memories (SRAM) 641. The second memories 641 store the weights corresponding to the PPG feature maps (LSTM W0 to LSTM W5) and the weights of the hidden states (LSTM V0 to LSTM V5). The long short term memory module 64 performs a gate parameter operation on the convolutional pooling data. This operation is conducted within each processing element array module 62 to generate the recursive data. The recursive data is then processed through activation functions, specifically sigmoid and tanh functions, to compute the final current state (Ct) and hidden state (Ht) values, which are subsequently stored in the state buffer. Notably, Ht can either be directed to the data memory for subsequent layer computations or be output for further analysis. A difference value between the predicted blood glucose value and a true blood glucose value is generated, the difference value is calculated by a loss function, specifically the Mean Square Error (MSE), and the weights of each layer of convolutional neural network accelerator module are iteratively adjusted based on the loss function.

The second processor 50 issues an instruction to reset the data memory module 61, clearing residual data. Subsequently, the feature map is provided and stored in the data memory module 61. In the next step, the slave module is assigned, and weights are provided and stored in the SRAM within the slave module. After specifying the read address and configuring the hyperparameters of the layer, the chip begins computation. If all model computations are completed, the output can be transmitted back to the second processor 50 for analysis. If not, the flow returns to the slave module assignment step, and the next layer's computation is executed until the entire inference process is completed. Additionally, the chip can output the results of each layer to test the correctness of the computing function by setting the output answer signal to high.

The long short term memory module 64 allows dynamic adjustment of the model by permitting the number of input channels to vary between 1 and 10, catering to different application scenarios. Given the maximum number of weights allowed in SRAM (256 words) and a maximum limit of 10 for the long short term memory module 64 kernel count, the size of the input feature map can vary across 64 different configurations, ranging from 1 to 64. Users can configure the architecture of the long short term memory module 64 according to their specific requirements. Users transmit model architecture parameters and corresponding weights to the configurable the long short term memory module 64 via the APB BUS. To efficiently control the second memories 641 write operations for input weights (weight_{w}) and state weights (weight_{y}), global address_{w} and global address, are separately used to inform the starting position for all SRAM_{weight} write operations. The decoding of Paddr received through the APB BUS determines whether WEN should be set to 0, and each second memory 641 weight corresponds to the weights required for one channel of the input feature map.

Simultaneously, considering the efficient forward computation and avoiding unnecessary additional address control circuitry, the read behavior of SRAM_{weight} also adopts a global address approach to inform the entire the second memories 641 of the read position. Therefore, the forward propagation of weights is designed with the concept based on 10 channels. Before the multiplication operation of input Xₜ and weights W_{f,i,g,o}, AND and OR logic is employed to determine whether the current channel input data is valid. If it is a valid input, normal computation is performed; if it is an invalid input, the result is directly set to 0.

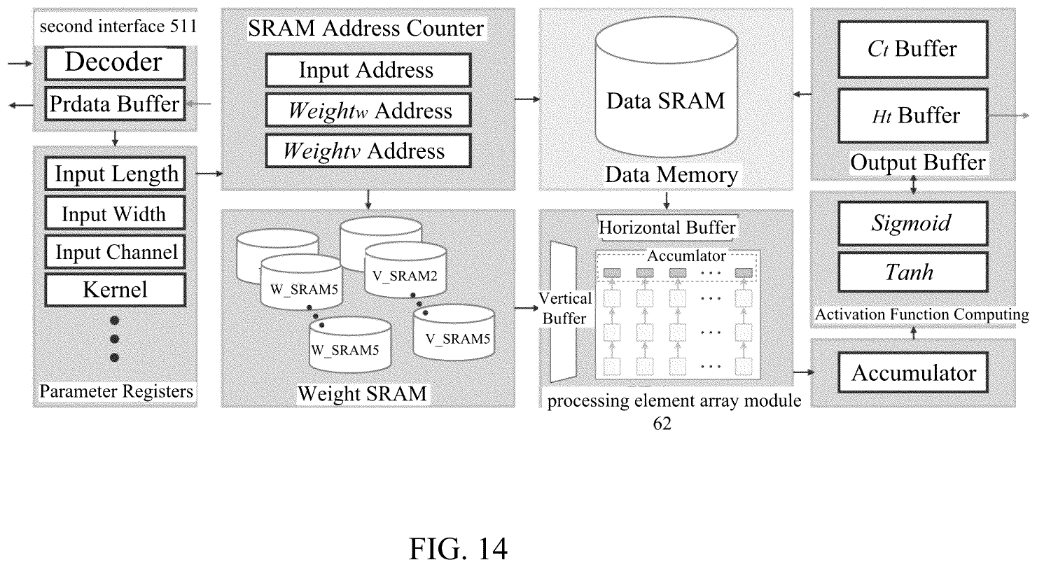

Please refer to FIG. 14, FIG. 14 is a schematic diagram of the long short term memory network module of the present invention. Under the condition of a maximum of 10 channels input, there is at least 1 channel input data. If OR (channel [3:1]) is high, it indicates that the input channel is at least 2. If OR (channel [3:2]) is high, it signifies that channel 3 is valid since the input channel is at least 4. If channel [3] is high, it means that there is at least input channel 8. The detailed numbers of channel profiles can be traced in FIG. 14 by using only basic “OR” and “AND” logic cells for implementation. For the multiplication operation between the previous hidden state hₜ₋₁ and the weights V_{(i,f,g,o)}, the same method is employed to determine the effective number of kernels for the long short term memory module 64 based on user input. If it is an effective kernel, normal computation is performed; otherwise, the result is set to 0.

The vector inner product operation for obtaining A_{i,f,g,o} utilizes a partial Multiply-Accumulate (MAC) structure within the processing element array module 62. The data flow for calculating a single-channel data A_{Ni,f,g,o} within the processing element array module 62, where N represents the channel number (1, 2, . . . , 10). In the horizontal direction, reusable feature map inputs are broadcasted horizontally and multiplied with the corresponding weights. The order in which weights are read from SRAM, requiring a total of 4 cycles to complete reading one set of W_{f,i,g,o}. Before obtaining a sufficient number of weights, the accumulation of values in the processing element array module 62 remains unchanged, using zero-padding. Once each processing element array module 62 completes the computation for one LSTM unit result A_{Ni,f,g,o}, the output of the processing element array module 62 is fed into an external accumulator to calculate the final A_{i,f,g,o}.

The activation function also takes into account hardware-friendliness, and the General Sigmoid function involves exponential operations to project the input into the range (0, 1). The sigmoid function approximates this behavior using two linear functions, and the approximation can be implemented through register shifting, comparators, and adders, rather than using exponential IP(ex). Despite a slight sacrifice in accuracy, it offers improved power and area efficiency. Based on the same design considerations, the other activation function, tanh, is implemented using the Isosceles Triangular Approximation instead of directly relying on exponential IP.

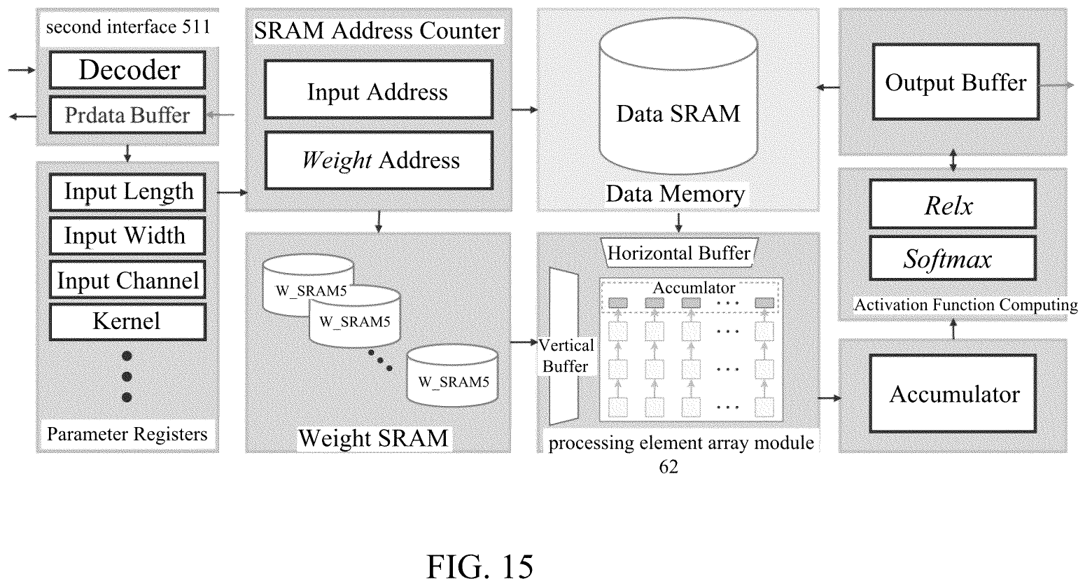

Please refer to FIG. 15, FIG. 15 is a schematic diagram of the matrix-vector product unit of the present invention. In a preferred embodiment, the present invention includes a matrix-vector multiplication module 65, which achieves matrix-vector multiplication computations for the fully connected layer by controlling the processing element array module 62. Initially, the second interface 511 transmits parameters for the Fully Connected layer, including the size of the input feature map (Ifmap), the number of output neurons, and information about whether it is the final layer, via the APB BUS. Subsequently, the Iteration Computing Control orchestrates the counter to sequentially read from and input the processing element array module 62 for computation. After the activation function is applied, the computation results are stored back in Data Memory or transmitted through the APB BUS, based on the layer parameter judgment.

The computation data flow adopts a strategy to efficiently reuse data read from SRAM. The input feature map (Ifmap) is horizontally broadcasted with the weight that has the lowest reuse degree. The Ifmap is broadcasted to the entire processing element array module 62 in three clock cycles. In the first two clock cycles, zero values are input to ensure that weights are not computed together. In the first two clock cycles, zero values are input to ensure that weights are not computed together. In the third clock cycle, the data (x1, x2, x3) is input, and the process continues for the next set of three data inputs in the next iteration. Due to the design size limitation of the processing element array module 62, the input Ifmap needs to be divided into groups of three for computation. Vertical input is implemented using a systolic array approach, where in each clock cycle, the weights shift by one row, corresponding to the top row of Ifmap in the processing element array module 62. Because of the design size limitation of the processing element array module 62, one row of weights contains 10 values, so weights are also divided into groups of 10 for computation. The output from the top row of the 3×10 processing element array module 62 is only available after the entire Ifmap has been processed once and the accumulation is complete, resulting in the output of 10 neural results. For neural networks with more than 10 neurons, the next iteration will involve rereading Ifmap and the corresponding weights to continue the computation.

In the matrix-vector multiplication module 65, the activation functions for the fully connected layer include the ReLU and Softmax functions. When the fully connected layer receives instructions indicating that the current layer computation is not the final layer, it adopts the ReLU activation function. If it is the final layer corresponding to the output classification layer, the activation function becomes Softmax. The result of the ReLU function can be directly determined through the signed bit and outputted to the data SRAM.

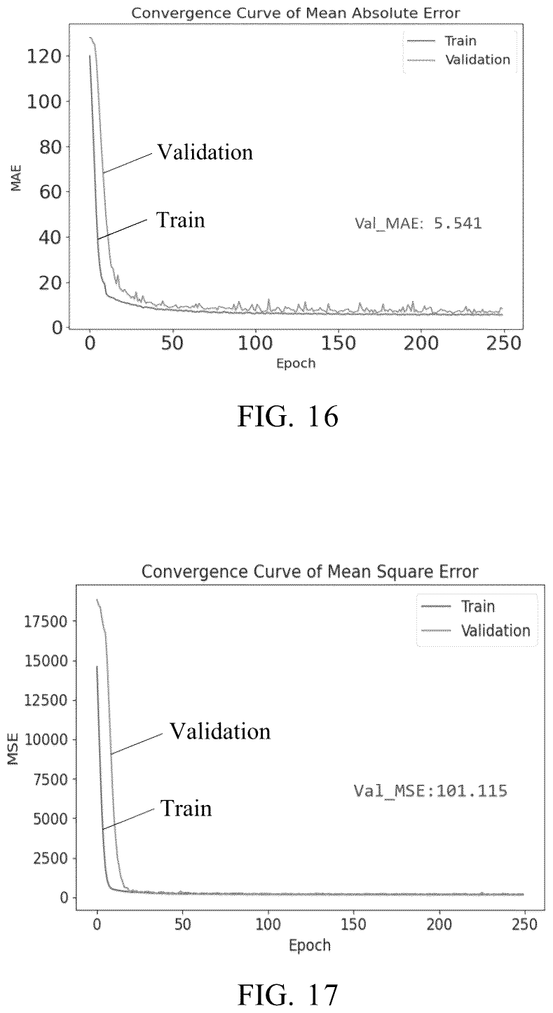

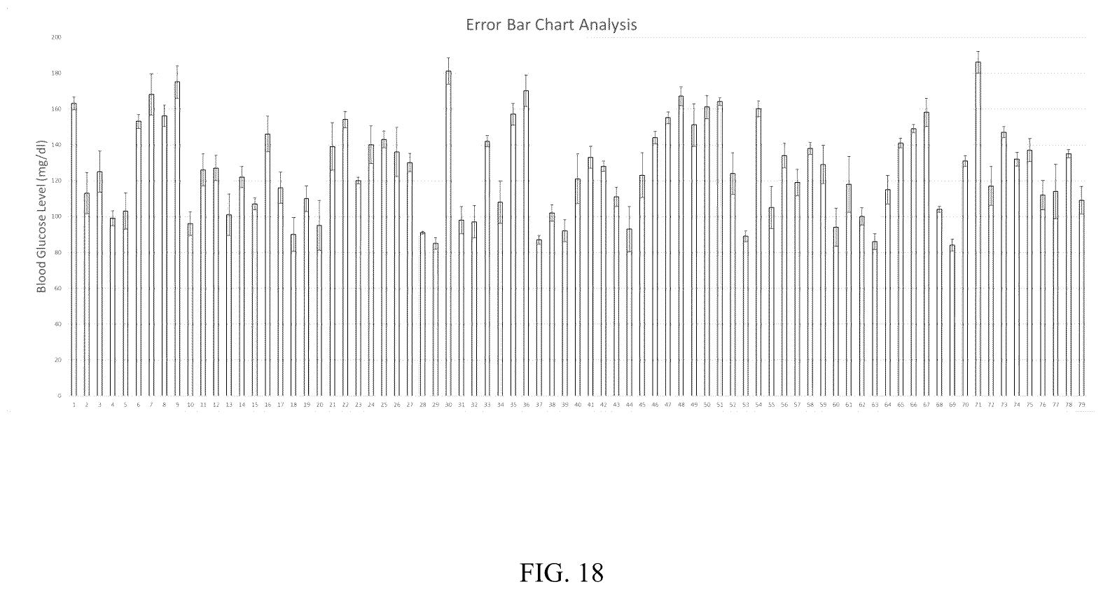

Please refer to FIG. 16 and FIG. 17, FIG. 16 is a curve chart of mean absolute error within 250 cycles of the present invention; and FIG. 17 is a curve chart of the mean square error within 250 cycles of the present invention.

As shown in FIG. 16 and FIG. 17, the dataset is partitioned into three subsets: 70% for training, 20% for validation; and the remaining 10% for testing. The Mean Absolute Error (MAE) and Mean Squared Error (MSE) curves are depicted over 250 epochs, with resulting MAE and MSE values of 5.541 and 101.115 on the validation data. MAE represents the mean of the absolute differences between predicted and reference values, and MSE includes an additional step of squaring the loss before averaging. Due to the inclusion of squared error calculations, MSE tends to provide a more reliable measure of model performance, as it accounts for larger errors more significantly. In contrast, MAE calculates the average loss without amplifying the impact of individual large errors, offering a different perspective on model evaluation. The evaluation of model accuracy involves regression analysis, comparing the predicted Blood Glucose Level (BGL) against the reference BGL. The correlation between the predicted and reference values is quantified through the calculation of the R-squared value. The resulting R2 value of 0.883 attests to a robust positive correlation, indicative of high model performance.

Please refer to FIG. 18, FIG. 18 is an error bars analysis between predicted blood glucose level concentrations and reference blood glucose concentrations on the test data of the present invention. According to FIG. 18, the ISO 15197:2013 standard is applied to evaluate whether the model complies with established standard.

According to ISO 15197:2013, the tolerance error requirements are as follows: for BGL values smaller than 100 mg/dl, at least 95% of the results should fall within ±15 mg/dl of the traceable laboratory method. For BGL values greater than or equal to 100 mg/dl, the acceptable tolerance is at least 95% of the results within ±15%. In the error bar results, the largest standard deviation observed for BGL values higher than 100 mg/dl is ±15.59 mg/dl for a BGL of 118 mg/dl, well within the acceptable error range of ±27 mg/dl. Similarly, the largest standard deviation for BGL values smaller than 100 mg/dl is ±13.86 mg/dl for a BGL of 95 mg/dl, fitting comfortably within the acceptable error range of ±15 mg/dl.

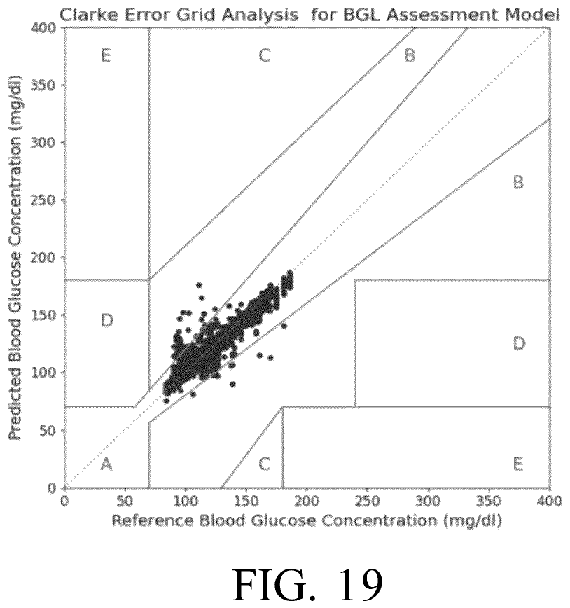

Please refer to FIG. 16 and FIG. 19, FIG. 19 is a Clarke error grid analysis of the blood glucose evaluation model of the present invention. Another requirement of the ISO 15197:2013 criteria is that at least 99% of the results should fall within zones A and B in a consensus error analysis. To test this standard, Clarke Error Grid Analysis is utilized to analyze the predicted BGL versus reference BGL. The Clarke Error Grid is divided into five regions, each delineating the potential impact on accurate treatment. Region A represents predicted values that align closely with the reference values, within a 20% error of the reference value. Region B signifies results that are acceptable, falling outside a 20% error of the reference value, but still within a safe margin. Region C indicates predicted results that might lead to unnecessary treatment, especially when the predicted value is opposite to the reference value within the healthy range. Region D denotes potential dangerous failures to detect hypoglycemia or hyperglycemia, while Region E suggests points that could cause confusion in treatment between hypoglycemia and hyperglycemia. For an effective self-blood glucose monitoring device, the result points should ideally map to Region A and Region B, ensuring accurate and safe blood glucose management for or individuals with diabetes. In FIG. 19, the experimental results are mapped on the Clarke Error Grid. Almost all points are located in Region A and Region B, signifying that the BGL assessment model exhibits potentially accurate performance.

In order to enable the objective, technical features and advantages of the invention to be more understood by a person skilled in the art to implement the invention, the invention is further illustrated by accompanying the appended drawings, specifically clarifying technical features and embodiments of the invention, and enumerating better examples. In order to express the meaning related to the features of the invention, the corresponding drawings herein below are not and do not need to be completely drawn according to the actual situation.

What is claimed is:

1. A signal quality assessment system based on photoplethysmography, comprising:
a database including a blood glucose level dataset and a PPG signal dataset, the blood glucose dataset including at least one blood glucose value, and the PPG signal dataset including at least one PPG signal obtained through a PPG sensor detecting light absorption and reflection rates from blood of a subject;
an AI accelerator platform communicatively connected to the database through a Bluetooth module, the AI accelerator platform comprising:
a first processor communicatively connected to the database and obtaining a training dataset from the blood glucose dataset and a PPG signal dataset;
a preprocessing engine communicatively connected to the first processor through a first interface and extracting multiple PPG feature maps from the PPG signal in the training dataset;
an AI module comprising multiple layers of convolutional neural network and communicatively connected to the first processor through the first interface, the AI module trained based on the PPG feature maps using a template matching method and a peak detection method to extract multiple pulse waveforms from the PPG feature maps, and to established a template PPG signal to calculate a correlation coefficient between the template PPG signal and the pulse waveform, and wherein each time the AI module is trained to generate the correlation coefficient, a quality classification label, and an initial training model, and the correlation coefficients are averaged to obtain a threshold value, when the correlation coefficient is greater than or equal to the threshold value, the training of the artificial intelligence model is completed to generate a PPG signal quality assessment module, the quality classification label and the PPG signal quality assessment module are transmitted to the first processor through the first interface; and
an electronic device communicatively connected to the first processor through a transmitter, and configured to display the quality classification labels including vectors [0,1] and [1,0], and the PPG signal quality assessment module.

2. The signal quality assessment system according to claim 1, wherein the preprocessing engine includes a bandpass filter and a data normalization calculator, the bandpass filter filtering the PPG signal in the range of 0.5 Hz to 5 Hz to eliminate noise interference outside the frequency range and an external high-frequency noise to produce bandpass-filtered data, and the data normalization calculator normalizing the bandpass-filtered data input every 5 seconds using following equation 1, where x[n]ϵX and n=1, 2 to 320,
$$y [ n ] = \frac{x [ n ] - min ( x )}{max ( x ) - min ( x )} . \qquad equation 1$$

3. The signal quality assessment system according to claim 1, wherein the timelines from both the blood glucose level dataset and the PPG feature maps were extracted and compared to align at the same time points through the template matching method, and blood glucose values were retained after alignment, and the corresponding PPG signals were selected for one-minute records.

4. The signal quality assessment system according to claim 2, wherein the extracted one-minute PPG feature maps are subdivided into 5-second windows, the pulse waveform within a window is extracted by employing the peak detection, the template PPG signal is constructed by averaging each pulse waveform within a window, and the correlation coefficient between the template PPG signal and the pulse waveform is calculated according to the following Formula 2,
$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} {\sum }_{i = 1}^{n} {( {y}_{i} - \overline{y} )}^{2}}} . \qquad equation 2$$

5. The signal quality assessment system according to claim 1, wherein a difference value between the quality classification label and a true label is generated, the difference value is calculated by a loss function, specifically a binary cross entropy, and the weights of each layer of convolutional neural networks are iteratively adjusted based on the loss function.

6. A blood glucose level prediction system, comprising:
a dataset including at least one PPG signal with a quality classification label represented by vector [0,1], generated by a signal quality assessment system based on photoplethysmography as described in any one of claim 1;
a second processor communicatively connected to the dataset through a second interface and obtaining a blood glucose training dataset from the dataset;
an edge AI accelerator communicatively connected to the second processor through the second interface to receive the PPG signals from the blood glucose training dataset to accomplish a blood glucose prediction result, and the edge AI accelerator comprising:
a data memory module storing the PPG signal and computed multiple PPG feature maps, and providing relevant instructions to the second processor for resetting or inputting data;
a processing element array module composed of multiple processing units, each processing unit including a multiply accumulate unit to handle convolution and matrix-vector multiplication operations, and to perform parallel processing of the PPG signal;
a convolutional neural network accelerator module including multiple convolutional layers, a pooling layer, a fully connected layer, and multiple first memories used to store weights required by the convolutional layers, the pooling layer, and fully connected layers, the PPG signal accelerated and processed in parallel through each processing unit to generate the PPG feature maps, the PPG feature maps processing through the activation function to a generate convolutional pooling data, which is then stored in the first memories for subsequent layer calculations, or sent back to the second processor for convolutional pooling data verification and analysis; and
a long short term memory module recursively processing the convolutional pooling data, in each processing unit, computing the convolutional pooling data to extract temporal features to generate recursive data, a regression prediction to be performed by the fully connected layer, the recursive data undergoes matrix-vector product computation in each processing unit to generate a predicted blood glucose value and a blood glucose prediction module, which the predicted blood glucose value and the blood glucose prediction module are transmitted to the second processor through the second interface; and
an electronic device communicatively connected to the second processor through a transmitter, and configured to display the predicted blood glucose value and the blood glucose prediction module.

7. The blood glucose level prediction system according to claim 6, wherein the long short term memory module includes multiple second memories storing the weights corresponding to the PPG feature maps and the weights of hidden states, the Long Short term Memory network module performs a gated parameter operation on the convolutional pooling data, and the operation is carried out in each processing unit to generate recursive data which is then processed through the activation functions to calculate the final current state (Ct) value and the hidden state (Ht) value.

8. The blood glucose level prediction system according to claim 6, wherein each convolutional layer includes at least one filter and uses a Rectified Linear Unit function as a first activation function, the fully connected layer includes at least one neuron and uses a linear function as the second activation function, and a regression prediction is performed through the fully connected layer.

9. The blood glucose level prediction system according to claim 8, wherein the linear function includes both the Rectified Linear Unit function and a Softmax function, when a preceding layer of the fully connected layer is not the final layer, the fully connected layer uses the Rectified Linear Unit function as the activation function, and when the fully connected layer is the final layer, the second activation function becomes Softmax, which the result of the Rectified Linear Unit function can be directly determined by utilizing signed bits to output to the first memories.

10. The blood glucose level prediction system according to claim 6, wherein a difference value between the predicted blood glucose value and a true blood glucose value is generated, the difference value is calculated by a loss function, specifically the Mean Square Error (MSE), and the weights of each layer of convolutional neural network accelerator module are iteratively adjusted based on the loss function.
